(19) United States
(12) Patent Application Publication
(10) Pub. No.: US 2005/0221421 A1
(43) Pub. Date: Oct. 6, 2005

(54) Method of producing and using heat shock proteins

(76) Inventors: Jean-Luc V. Tran, Camden, NJ (US); Charles W. Hewitt, Blackwood, NJ (US)

Correspondence Address: LICATA & TYRRELL P.C., 66 EAST MAIN STREET, MARLTON, NJ, 08053, US

(21) Appl. No.: 10/503,802

(22) PCT Filed: Feb. 7, 2003
(86) PCT No.: PCT/US03/03596

§ 371 Date: Apr. 5, 2005

Related U.S. Application Data
(60) Provisional application No. 60/355,769, filed on Feb. 8, 2002.

(51) Int. Cl. C12P 21/06; C07K 14/47

(52) U.S. Cl. 435/69.1; 530/350

(57) ABSTRACT

The present invention relates to a method of manufacturing and using heat shock proteins containing a step of initiating a coagulative necrotic process in a tissue. In particular, a method of manufacturing and using heat shock proteins comprises the steps of heating a tissue to initiate the coagulative necrotic process for a period of time, cooling the tissue, incubating the tissue in an appropriate growth medium, and collecting the supernatant from the tissue. In a preferred embodiment, the coagulative necrotic process is initiated by heating the tissue to 60°; or more. The tissue is then cooled to room temperature before incubating the tissue in growth media for 48 hours or less. Heat shock proteins produced in accordance with this method may be complexed to peptides or antigens to produce autologous vaccines in the prevention and treatment of various diseases. Furthermore, heat shock proteins may be used in diagnostic assays for various autoimmune and inflammatory diseases. Heat shock proteins produced in accordance with this method may also be used to quantify antibody levels that serve as disease markers.

### FIELD OF THE INVENTION

The present invention relates to the field of molecular biology, immunology, disease prevention and treatment, and more particularly, a method of producing and using heat shock proteins.

### BACKGROUND OF THE INVENTION

Heat shock proteins are an unusual group of highly conserved proteins that are produced by cells in response to a variety of stresses. Stressful conditions that induce cells to produce heat shock proteins include environmental changes, injury, disease, nutrient deprivation, inflammatory responses, oxygen radicals, toxins, viral and bacterial infection, and even behavioral or psychological stress. Upon exposure to stress, three distinct events take place in the cell: (1) the transcription of heat shock peptide mRNA is increased, while the transcription of most other mRNA is suppressed; (2) heat shock mRNAs are preferentially translocated to the cytoplasm; and (3) the heat shock proteins are preferentially translated by the ribosomes. The presence of heat shock proteins allows cells to endure through periods of stress by conserving physiological homeostasis and thus extending stress tolerance. For that reason, heat shock proteins are vital to the survival of cells in vulnerable situations.

Heat shock proteins also play a major role in cellular function in its basal state by maintaining normal homeostasis. Among its many functions, heat shock proteins facilitate the synthesis of new proteins and the disassembly and disposal of damaged proteins. Heat shock proteins also control the quality of proteins by helping newly synthesized polypeptides assemble into their proper conformation. Specifically, heat shock proteins “chaperone” the post-translational assembly by ensuring that polypeptides are properly folded and assembled into their oligomeric structures. Thus, heat shock proteins prevent the formation of “improper” structures that result from the exposure of hydrophobic or charged surfaces either within or between polypeptide chains.

Heat shock proteins are classified in families according to their molecular weight in kilodaltons (kDa). The major heat shock families include, but are not limited to HSP100, HSP90, HSP70, HSP60, HSP40, and sHSP, a family of small heat shock proteins that range from 12 to 30 kDa. The function and structure of heat shock proteins vary drastically between families and there is continuing research to identify new families and isoforms of heat shock proteins. For example, the heat shock protein 90 (HSP90) family includes various isoforms: HSP82, HSP83, and HSP89. The differences between these isoforms have yet to be elucidated.

As a family, the HSP90 proteins are the most abundant protein in the cytosolic fraction. Even though HSP90 expression is induced by stress, its constitutive level in many cells may be as high as 1-2% of the extractable cellular protein. The relatively high concentration of HSP90 in many cell types suggests that HSP90 may also play a general role in the cell, but little is known about its actual function. It has been suggested that HSP90 may function through protein-protein interactions as a molecular chaperone. Heat shock protein 70 (HSP70), like the HSP90 proteins, also functions as a molecular chaperone by interacting with the cellular proteins in an ATP-dependent manner. HSP70 expression, however, is induced by stress and is believed to be a diagnostic marker for stress. Heat shock protein 60 (HSP60) is synthesized both constitutively and in response to stress. It is believed that HSP60 is present in all species and that has a remarkable sequence homology among bacteria, plants and mammals. HSP60 is also believed to interact with various proteins during translocation and folding. The small heat shock protein family (sHSP) is a diverse group proteins that are characterized by the presence of conserved sequences of 80-100 residues. Six sHSPs have been identified in humans, including heat shock protein 27 (HSP27), which is believed to facilitate cellular recovery from cytoskeletal disruption resulting from cellular stress.

Ongoing research continues to reveal the diversity of heat shock protein function. It is now known that heat shock proteins have a significant role in development, immune response, regulation of gene expression, the regulation of translation, and inhibition of apoptosis.

In addition to its role in maintaining cellular homeostasis, heat shock proteins have been implicated in a variety of autoimmune and inflammatory diseases, such as insulin-dependent diabetes mellitus, rheumatoid arthritis, sclerodoma, mixed connective tissue disease, atherosclerotic lesiouns, carotid atherosclerosis, systemic lupus erthematous, temporo-mandibular joint syndrome, to name a few. Significantly higher levels of heat shock protein antibodies have been reported in patients with autoimmune and inflammatory diseases. For example, patients with atherosclerotic lesions were also found to have significantly higher levels of antibodies to HSP60 as compared with healthy patients without atherosclerotic lesions. Xu Q, et al. “Association of Serum Antibodies to Heat Shock Protein 65 with Carotid Atherosclerosis.” Lancet. 1993; 5(6):803-814. Immunoreactivity to HSP60 was also reported to play an important role in the development of insulin-dependent diabetes mellitus. Jones D B, et al. “Heat shock protein 65 as a β cell antigen of insulin-dependent diabetes.” Lancet. 1990; 336:583-585. Specifically, patients with insulin-dependent diabetes mellitus were found to have significantly higher levels of antibodies to HSP60 as compared to healthy patients. Ozawa, Y., et al. “Detection of Autoantibodies to the Pancreatic Islet Heat Shock Protein 60 in Insulin-dependent Diabetes Mellitus.” J. Autoimmun. 1996; 9:517-524.

In addition to autoimmune disease, heat shock proteins have been associated with rejection of tissue or organ transplants. The increased expression of heat shock protein in the allograft tissue or organ induces heat shock reactivity to promote the development of acute and chronic rejection. The precise role of heat shock proteins in the rejection of tissue or organ transplants, however, is yet to be determined.

Heat shock proteins have also been reported to interfere with cancer treatment by conferring drug resistance to cells. Studies have linked drug resistance to elevated levels of heat shock proteins in cells. For example, the increased expression of heat shock protein 70 (HSP70) in drug resistant cells has been found to affect the efficiency of combined hyperthermia and anti-cancer drug treatment. Brozovic, et al. Induction of Heat Shock Protein 70 in Drug-Resistant Cells by Anticancer Drugs and Hyperthermia. Neoplasma 2001; 48(2): 99-103. Also, the increased expression of HSP27 and HSP70 has been correlated to drug resistance in breast cancer patients treated with combination chemotherapies. Vargas-Roig L M, et al. Heat Shock Protein Expression and Drug Resistance in Breast Cancer Patients Treated with Induction Chemotherapy. Int. J. Cancer 1998; 79(5): 468-75. Thus, the association between the elevated levels of heat shock protein and drug resistance must therefore be taken into account in developing a chemotherapeutic plan for patients.

Heat shock proteins have also been observed to elicit cellular immunity against tumor cells. Further studies have revealed that heat shock proteins isolated from tumor cells were typically bound to antigenic peptides specific to that tumor cell and responsible for activating the T cell response in the host. Thus, the specific immunogenicity of heat shock proteins was attributed to the unique repertoire of antigens with which it formed a complex in the tumor.

These observations gave rise to the idea of using heat shock proteins as vaccines to prevent and treat cancer. Studies have revealed that heat shock proteins were capable of eliciting an immune response because of their association with antigenic peptides. In cells, heat shock proteins are associated with a broad spectrum of peptides, polypeptides, proteins, and antigens with which they form complexes (“HSP-complexes”). Beckman et al. “Interaction of HSP70 with Newly Synthesized Proteins: Implications for Protein Folding and Assembly.” Science. 1990; 248:850-854. HSP-complexes are also formed to transport antigenic peptides to the MHC class I molecule on the cell's surface for antigen presentation. Srivastava et al., “Heat Shock Protein—Peptide Complexes in Cancer Immunotherapy.” Current Opinion in Immunology. 1994; 6:728-732.

Accordingly, heat shock proteins also have potential therapeutic applications to disease prevention and treatment. In recent years, heat shock proteins have become object of intense work by scientists in the prevention and treatment cancer, tuberculosis, Alzheimer's disease, AIDS, genital herpes, and diabetes, among many others. The use of heat shock proteins as a vaccine is a new approach to disease prevention and treatment. This is particularly promising for diseases, such as cancer, where the success rates are often dismal under conventional treatment plans.

The immunogenicity of HSP-complexes has applications in the vaccination against cancers and infectious diseases. For cancers, HSP-complexes isolated from a patient's own cancer can serve as customized, patient-specific therapeutic vaccines. The use of HSP-complex vaccines in infectious diseases provides an opportunity to complex known antigens or peptides with HSPs and use these complexes to elicit a peptide-specific CTL response, in spite of their exogenous presentation. Heat shock proteins can also be purified from cells infected in vitro with viruses. Another way to activate cellular immunity is to complex the heat shock protein to defined antigens that are synthetically manufactured. And yet other methods of eliciting antigen-specific cellular immunity against tumors have been proposed using endogenous, synthetic, bacterial, and viral antigens.

The vast diversity of heat shock proteins and their role in cellular development and homeostasis, immune response, disease, and disease treatment make them particularly valuable in research and medical treatment. Although heat shock proteins commercially available, they are typically available only in small quantities and at exorbitantly high costs. Moreover, commercially available heat shock proteins are typically produced by recombinant animal protein purified from a bacterial system. Furthermore, most commercially available heat shock proteins can only be used for research and not for diagnostic or therapeutic purposes. Accordingly, there is a need for an inexpensive method of manufacturing large quantities of native human and animal heat shock proteins for use in research, medical treatment, and diagnostic assays.

### SUMMARY OF THE INVENTION

The present invention satisfies a long-felt need for a method for producing large quantities of heat shock proteins from tissue.

Coagulative tissue necrosis may be initiated by a variety of methods including, but not limited to, exposing the tissue to irradiation, thermal injury, hypoxia, and toxins. Generally, necrosis refers to a sequence of morphologic changes that follow cell death in living tissue. Coagulative necrosis is a process characterized by cell swelling, denaturation of cytoplasmic proteins, break-down of cell organelles, and complete ultimately cellular and tissue death. Accordingly, it would be expected that heat shock proteins would be completely denatured upon the initiation of coagulative tissue necrosis.

Surprisingly, an unexpectedly high quantity of heat shock proteins are produced as a result of initiating coagulative necrotic changes in tissue. The production of heat shock proteins is significantly increased upon the initiation and progression of coagulative necrosis until complete cellular and tissue death occurs.

In one embodiment of the present invention, coagulative necrosis is initiated by by heating tissue at a defined temperature and duration sufficient to induce the production of heat shock proteins, cooling the tissue, incubating the tissue in an appropriate growth medium, and collecting the supernatant containing the heat shock protein from the tissue. One method of initiating coagulative necrosis is to heat the tissue to a temperature of 60° to 80° C. An increase in temperature results in a more advanced coagulative necrotic state in the tissue and an unexpected increase in the production of heat shock proteins.

In a particularly preferred embodiment, the tissue is heated to 80° C. for 10 seconds and cooled to room temperature before incubating the tissue in growth media for 48 hours.

In another embodiment of the present invention, the tissue may be subject to stress by incubating it in a bioreactor, such as a microgravity culture system provided by the rotating cylindrical cell culture vessel (RCCS bioreactor) developed from the NASA space program and commercially available from Synthecon, Inc. (Houston, Tex.). It has been shown that tissue incubated in the RCCS bioreactor reproducibly leads to cellular apopotosis. Similarly, tissue may be incubated at a temperature sufficient to induce the production of heat shock proteins in the RCCS bioreactor.

The heat shock protein families and isoforms produced in accordance with the present invention may be purified by a number of methods, including but not limited to centrifugation, electrophoresis, and chromatographic methods, including but not limited to, gel filtration, ion exchange, and chromafocusing, immunoaffinity, hydrophobic interaction, reverse phase HPLC, gel electrophoresis, centrifugation, and affinity chromatographic techniques.

Heat shock proteins have applications for use as vaccines in the prevention and treatment of various diseases. Accordingly, in one embodiment, heat shock proteins produced in accordance with the present invention may be associated with a peptide or antigen to produce an HSP-complex. The HSP-complex in accordance with this present invention may then be purified by ADP-affinity, heparin-agaorose, or other methods that will keep in tact the association between the heat shock protein and antigen or peptide in the HSP-complex.

In yet another embodiment of the present invention, heat shock proteins produced in accordance with the present invention may be used in a diagnostic assay to quantify the level of antibodies present in a patient as a marker for disease. This assay may be used where a correlation has been found between the increased antibody level for a heat shock protein and a particular disease, such as various autoimmune and inflammatory diseases.

### DESCRIPTION OF THE DRAWINGS

FIG. 1 is a Western Blot of heat shock proteins from neonatal foreskin tissue that was minced, heated at 80° C. for 10 seconds and incubated at 37° C. for 48 hours.

FIG. 2 is a Western Blot comparing the production of HSP70 by neonatal foreskin heated at 80° C. for 10 seconds at incubation times of 24, 48, and 72 hours in lanes 2, 3, and 4 (from left to right), respectively with unheated neonatal foreskin at incubation times of 24, 48, and 72 hours in lanes 5, 6, and 7, respectively. The marker is depicted in lane 1.

FIG. 3 is a Western Blot comparing the HSP70 produced by neonatal foreskin in lanes 1-3 (from left to right) and recombinant HSP70 of known, increasing concentrations in lanes 4-11. The concentration of recombinant HSP70 is as follows: lane 4 (70 ng/ml), lane 5 (140 ng/ml), lane 6 (281 ng/ml), lane 7 (0.56 μg/ml), lane 8 (1.125 μg/ml), lane 9 (2.5 μg/ml), lane 10 (5 μg/ml), and lane 11 (10 μg/ml). The heat shock protein markers are shown in lane 12.

FIG. 4 is a Western Blot of HSP70 produced in accordance with the present invention. From left to right, lane 1 is the heat shock protein marker, lanes 2, 3, 4 and 5 are HSP70 produced by heating neonatal foreskin at 90° C., 80° C., 70° C., and 60° C., respectively, for 10 seconds followed by 48 hours of incubation time, lanes 6, 7, 8, and 9 are HSP70 produced by heating neonatal foreskin at 90° C., 80° C., 70° C., and 60° C., respectively, for 10 seconds followed by 24 hours of incubation time.

### DETAILED DESCRIPTION OF THE INVENTION

Definitions

“Antigen” refers to any substance that is capable of eliciting an immune response.

“Heat shock protein” or “HSP” refers generally to a large family of stress response proteins and their isoforms existing constitutively in the cell at its basal state and/or inducibly in the cell in response to stress, including but not limited to all isoforms of heat shock protein in the HSP100, HSP90, HSP60, HSP40, and sHSP families.

“HSP-complex” as used herein, refers generally to a heat shock protein that is associated with a peptide or an antigen.

“Peptide” as used herein, refers to a series of two or more peptides and includes polypeptides, proteins, protein sequences, amino acid sequences, denatured proteins, oncogenes, and portions of oncogenes.

“Tissue” as used herein refers to any group of similar cells that are united to perform a particular function and includes, but is not limited to, organs, skin, brain, heart, lung, liver, spleen, pancreas, thymus, thyroid, lymph node, stomach, kidney, bladder, intestine, colon, testis, mammary, ovary, uterus, muscle, bone, and any artificial equivalent thereof.

Introduction

In one embodiment of the present invention, heat shock proteins are produced by treating the tissue in a manner to initiate coagulative necrotic changes. Coagulative necrotic changes are initiated by energy transfer, which can be a function of temperature and time, among other factors. Accordingly, coagulative tissue necrosis may be initiated by a variety of methods including, but not limited to, exposing the tissue to irradiation, thermal injury, hypoxia, and toxins.

In a particularly preferred embodiment, the coagulative necrotic changes are initiated by heating a tissue at varying temperatures and duration to induce the production of heat shock proteins, cooling the tissue, incubating the tissue in the appropriate growth medium, and collecting the supernatant containing the heat shock protein from the tissue.

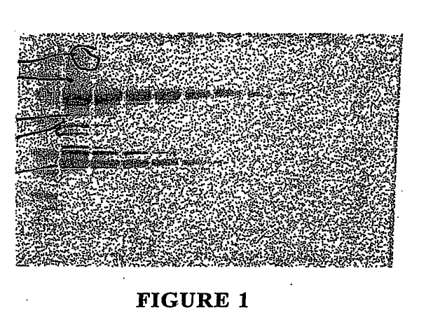

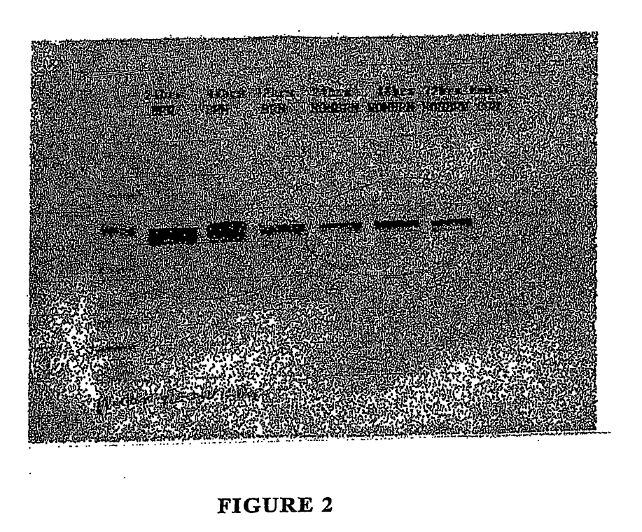

One method of initiating coagulative necrosis to heat the tissue at extremely high temperatures. It has been demonstrated that the production of heat shock proteins in neonatal foreskin is unexpectedly and significantly high when heated to 60° to 80° C. Accordingly, in a preferred embodiment, the tissue is heated to 80° C. for 10 seconds and cooled to room temperature before incubating the tissue in growth media for 48 hours. The Western Blot of FIG. 1 shows the bands of heat shock proteins produced under these conditions. FIG. 2 further demonstrates the marked difference in the production of HSP70 by tissue heated at 80° C. with the production of HSP70 by unheated tissue at varying incubation times.

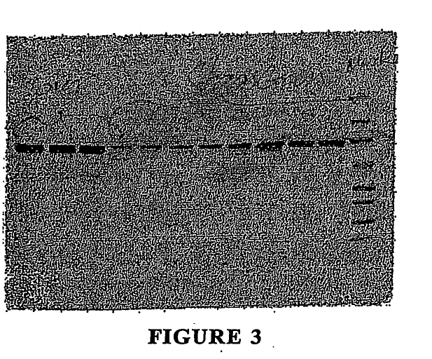

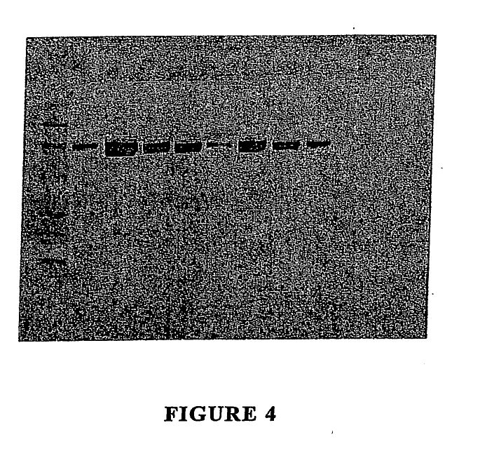

The significantly high quantity of heat shock proteins produced in accordance with this invention is further demonstrated by the Western Blots of FIG. 3. An increase in temperature results in a more advanced coagulative necrotic state in the tissue and an unexpected increase in the production of heat shock proteins. However, the production of heat shock protein markedly decreases as the tissue approaches complete necrosis. FIG. 4 illustrates the increase in heat shock proteins as a function of the coagulative necrotic state of the tissue. FIG. 4 also shows a decreased production of heat shock protein as the tissue approaches complete necrosis at 90° C.

In another embodiment of the present invention, the tissue may be subject to stress by incubating it in a bioreactor, such as a microgravity culture system provided by the rotating cylindrical cell culture vessel (RCCS bioreactor) developed from the NASA space program and commercially available from Synthecon, Inc. (Houston, Tex.). It has been shown that tissue incubated in the RCCS bioreactor reproducibly leads to cellular apopotosis. Similarly, tissue may be incubated at a temperature sufficient to induce the production of heat shock proteins in the RCCS bioreactor.

The various heat shock protein families may be separated and purified by using a combination of centrifugation, electrophoresis, and chromatographic methods, including but not limited to, gel filtration, ion exchange, and chromafocusing, immunoaffinity, hydrophobic interaction, reverse phase HPLC, gel electrophoresis, centrifugation, and affinity chromatographic techniques, and other chromatographic techniques known to those of ordinary skill in the art. In one embodiment, heat shock protein families and isoforms may be purified by using ion-exchange chromatography followed by affinity chromatography on ATP-agarose. In another embodiment of the present invention, the families and isoforms of heat shock proteins may be isolated using heparin-agarose chromatography. In yet another embodiment of the present invention, families of heat shock proteins may be purified by using a one-step gelatin-agarose method.

In another embodiment of the present invention, the heat shock proteins produced in accordance with the present invention may be complexed to a peptide or an antigen to produce a HSP-complex and used as a vaccine in the prevention and treatment of various diseases, such as cancer and various infectious diseases. HSP-complexes have immunological significance as vaccines because they are capable of inducing powerful immune responses against the peptides or antigens in the HSP-complexes. In another embodiment, heat shock proteins produced in accordance with the present invention may be complexed to defined antigens that are synthetically manufactured. In yet another embodiment, heat shock proteins may be complexed with endogenous, synthetic, bacterial, and viral antigens. A method for the de novo production of HSP-complex would allow the large-scale production of vaccines without the need for removing tissue from the patient. Also, it permits the large scale production of prophylactic vaccines against a wide variety of diseases by using peptides that represent known oncogenic mutations or peptides representing portions of known viral proteins. The HSP-complex produced in accordance with this present invention may then be purified by ADP-affinity, heparin-agarose, or other purification methods that will keep in tact the association between the heat shock protein and antigen or peptide in the HSP-complex.

In yet another embodiment of the present invention, heat shock proteins produced in accordance with the present invention may be used in a diagnostic assay to quantify the level of antibodies present in a patient as a marker for disease. This assay would be used where a correlation has been found between the increased antibody level for a heat shock protein and a particular disease, such as various autoimmune and inflammatory diseases. For example, a specific heat shock protein is produced and purified in accordance with the present invention. A biological sample, such as patient sera, synovial or other bodily fluid is obtained and exposed to the heat shock protein under conditions that would allow the antibodies to bind to the heat shock protein. The antibodies may then be quantified by enzyme-linked immunosorbant assay (ELISA), radioimmunoassay (RIA), or other immunoassays known to those of ordinary skill in the art. This result is then compared to the results obtained from the same biological sample from normal patients.

The following descriptions are provided only as examples and should not be understood to be limiting on the claims. Based on the description, a person of ordinary skill in the art may make modifications and changes to the preferred embodiments, which does not depart from the scope of the present invention.

### EXAMPLE 1

### Method of Producing Heat Shock Protein

In one embodiment, the production of heat shock proteins was induced by initiating the coagulative necrotic changes in the tissue by subjecting it to extreme heat, cooling the tissue to room temperature, and then incubating the tissue in a growth medium for a specific period of time. In a particularly preferred embodiment, neonatal foreskin is heated to 80° C. for 10 seconds and the incubated for 48 hours at 37° C. in KGM2. Under these conditions, the neonatal foreskin induces the production of an unprecedented amount of heat shock proteins.

For example, neonatal foreskin obtained from circumcision was divided into two groups: Group I was subjected to extreme heat exposure in phosphate buffered saline (PBS) at 80° C. for 10 seconds, then cooled to room temperature to create a burned group (BRN). Group II served as a non-burned/stress control. Both skin from these two groups were cut into tiny pieces approximately 1-2 mm, then incubated at 37° C. in a supplemented keratinocyte growth medium (KGM2). Supernatants from each of these groups were collected at specific time intervals: 24, 48, 72 hours following burn and non-burn/stress. SDS-PAGE was performed on these groups, and silver staining was utilized for visualization of the proteins and molecular weight determination. KGM2 served as a control. Western Blots were performed with polyclonal antibodies to identify specific HSP 27, 40, 60, and 70 proteins. Western blots detected bands in high densities for HSPs 27, 40, 60, 70 proteins in both burned and non-burned/stress, but not in the controls. The Western blots depicted in FIG. 3 demonstrate the high density of the HSP70 band as compared with the bands of known HSP70 concentrations.

In another embodiment of the present invention, tissue may be subject to stress by culturing it in one of several culture systems. In a preferred embodiment, the production of heat shock proteins may be induced by incubating a tissue in a bioreactor, such as a microgravity culture system provided by the rotating cylindrical cell culture vessel (RCCS bioreactor) developed from the NASA space program, and commercially available from Synthecon, Inc. (Houston, Tex.). The RCCS bioreactor is described in detail in U.S. Pat. Nos. 5,763,279 and 5,437,998 and herein incorporated by reference as if fully set forth herein. It has been shown that incubation times of tissue in the RCCS bioreactor reproducibly leads to a predetermined amount of apoptosis. Similarly, the induction of heat shock proteins may be controlled by incubation of tissue in the RCCS bioreactor for a defined period of time.

### EXAMPLE 2

### Purification of Heat Shock Proteins

The various heat shock protein families may be separated and purified by using a combination of centrifugation, electrophoresis, and chromatographic methods, including but not limited to, gel filtration, ion exchange, and chromafocusing, immunoaffinity, hydrophobic interaction, reverse phase HPLC, gel electrophoresis, centrifugation, and affinity chromatographic techniques.

For example, heat shock proteins may be purified using a procedure employing DE52 ion-exchange chromatography followed by affinity chromatography on ATP-agarose. Welch W J, et al. “Rapid Purification of Mammalian 70,000-Dalton Stress Proteins: Affinity of the Proteins for Nucleotides.” Molec. and Cell Bio. 1985; 6:1229-1237.

In another example, 90, 72 and 73 kDa heat shock proteins may be purified by column chromatography, using a Whatman DEAE-cellulose column (1.5×20 cm) packed with DE52. The supernatant obtained from burned or stressed cells, as disclosed above, may be diluted with Buffer B (comprising 20 mM Tris-acetate, pH 7.6, 20 mM NaCl, 0.1 EDTA, 15 mM 2-mercaptoethanol) until the conductivity of the supernatant-Buffer B solution approaches that of the conductivity of Buffer B alone. After the DE52 column is equilibrated with this buffer solution and a baseline value for the absorbance at 280 nm (A₂₈₀) is established, the supernatant-Buffer B solution may be applied to the DE52 column. The DE52 column is then washed with Buffer B until the A₂₈₀ returns to its baseline value. The heat shock proteins bound to the column may then be eluted with a linear gradient of 20 to 500 mM NaCl. Fractions of heat shock proteins demonstrating a peak at A₂₈₀ may then be collected. The first peak fraction may contain the 72 and 73 kDa heat shock proteins and the third peak may contain 90 and 100 kDa heat shock proteins. These two fractions may be pooled separately and dialyzed extensively against Buffer C (comprising 20 mM potassium phosphate, pH 7.6, 0.1 mM EDTA, 15 mM 2-mercaptoethanol) for further purification.

Purification of HSP70

In one embodiment of the present invention, the 72 and 73 kDa heat shock proteins (collectively referred to as “HSP70”) may be isolated and purified by a combination of column chromatography and high performance liquid chromatography. The pooled fractions from the DE52 column containing the HSP70 proteins may be clarified after dialysis Buffer C and applied to a hydroxylapatite column (1.2×10 cm) equilibrated in Buffer C. The column may be washed with Buffer C until the A₂₈₀ returns to a base-line value. Proteins may then be eluted from the column with a linear gradient of potassium phosphate (pH 7.5). The fractions containing HSP70 are then pooled and concentrated by negative pressure dialysis against Buffer B. Once concentrated, the HSP70 solution may be applied to a Sephacryl S-300 column (1.2×100 cm). The combined fractions containing HSP70s may then be applied directly to a DE53 column (1.2×10 cm) equilibrated in Buffer B and the HSP70 proteins may be eluted from the column with a linear gradient of NaCl. The peak fractions are then pooled and concentrated by a negative pressure dialysis against Buffer B. The concentrated fractions containing HSP70 can be applied to a Sepharose 6B column (2.5×90 cm) equilibrated in Buffer B. The known affinity of the HSP70 for ATP will allow for the use of ATP-Sepharose in their purification. Homogeneity of the peptides will be assessed by sodium dodecylsulphate polyacrylamide gel electrophoresis (SDS-PAGE) and N-terminal amino acid analyses. Welch W J, et al. “Purification of the Major Mammalian Heat Shock Proteins.” J. Biol. Chem., 1982; 257:14949-14959.

Purification of HSP90

In another embodiment, the pooled fractions from the DE52 column containing the 90 kDa heat shock proteins (referred herein as HSP90) may be purified by first collecting the pooled fraction containing the majority of HSP90, centrifuging it at 10,000×g and dialyzing it against Buffer C. The supernatant may then be applied to a hydroxylapatite column (1.2×10 cm) equilibrated Buffer C. The column may be washed with Buffer C until the A₂₈₀ returns to a base-line value. The HSP90 proteins may be eluted with a linear gradient of potassium phosphate (pH 7.5). The fractions containing HSP90 may then be pooled and concentrated by negative pressure dialysis against Buffer B using a Micro-Pro-Dicon concentrator (Bio-Molecular Dynamics, Beaverton, Oreg.). The concentrated proteins may then be chromatographed on a Sephacryl S-300 column (1.2×100 cm) developed in Buffer B. A single peak of HSP90 may be eluted from the column.

Purification by Heparin-Agarose Chromatography

Alternatively, heat shock proteins may be purified in one step using heparin-agarose chromatography. Heat shock proteins, such as HSP40, HSP60, HSP70, HSP90, and GRP94, have been isolated from a single tumor sample in one step using heparin-agarose chromatography. Menoret, A. et al., “Purification of Multiple Heat Shock Proteins from a Single Tumor Sample.” J. Immunol. Methods. 2000; 237:119-130.

Accordingly, in one embodiment, a cell pellet is obtained and homogenized in 40 ml hypotonic buffer comprising 10 mM NaHCO₃, 0.5 mM PMSF, pH 7.0 by dounce homogenization. The supernatant may then be obtained by 100,000×g centrifugation. The sample buffer may then be changed to the heparin buffer, comprising 20 mM sodium phosphate, 2 mM MgCl₂, 0.5 mM DTT, pH 7.2, with PD-10 column (Sephadex G-25, Pharmacia Biotech., Piscataway, N.J.). The sample may be applied at room temperate on a 20-mL Heparin Sepharose (Pharmacia Biotech., Piscataway, N.J.) equilibrated with the heparin buffer and washed. The proteins may be eluted by a linear gradient from 0 to 1 M NaCl in heparin buffer.

In another embodiment, 86 and 84 kDa heat shock proteins (collectively referred to as “HSP90”) fractions eluted from the heparin column may be further pooled and purified by changing the buffer of the HSP90 containing fractions to HQ-A buffer (20 mM sodium phosphate, 1 mM EDTA, 200 mM NaCl, pH 7.4) using PD-10 column (Sepharex G-25, Pharmacia Biotech., Piscataway, N.J.) and may be applied on a HQ-porous “PLC column equilibrated with buffer HQ-A. The column may then be washed until the A₂₈₀ dropped to a baseline level. HSP90 may be eluted with a linear gradient from HQ-A buffer to HQ-B buffer (comprising 20 mM sodium phosphate, 1 mM EDTA, 600 mM NaCl, pH 7.4). The fractions may be collected and analyzed by SDS-PAGE and immunoblotting. Menoret, A. J. Immunol. Methods, 2000; 237:119-130.

Purification by Gelatin-Agarose Chromatography

In yet another example, heat shock proteins may be purified by using a gelatin-agarose method. Cell lysates containing heat shock proteins are mixed with gelatin-agarose beads to bind the heat shock proteins to the beads. ATP is then added to the beads to release purified heat shock proteins. Nandan et al., “A Rapid Single-Step Purification Method for Immunogenic Members of the HSP Family; Validation and Application.” J. Immunol. Method. 1994; 176:255-263.

### EXAMPLE 3

### Production of Heat Shock Protein-Peptide Complexes

Heat shock proteins are ubiquitous in cells, and selected heat shock proteins, such as members of the HSP90 and HSP70 families. Heat shock proteins are often associated in cells with a broad spectrum of peptides, polypeptides, denatured proteins and antigens with which they form complexes. Because such HSP-complexes may be useful in vaccines against cancers and infectious diseases, a method of purifying HSPs together with their associated peptides is also desirable.

The HSP-complexes have immunological significance as vaccines because the HSP-complexes are capable of inducing powerful antigen-specific CD8+ cellular response against the peptides in the HSP-complexes, but not against the HSP itself. Srivastava, P K. “Purification of Heat Shock Protein-Peptide Complexes for Use in Vaccination against Cancers and Intracellular Pathogens.” Methods: A Companion to Methods in Enzymology. 1997; 12:165-171. Accordingly, HSP-complexes may be extremely valuable for use as a vaccine in the prevention and treatment of disease, and there exists a need for a method of producing large quantities of HSP-complexes for the research, prevention and treatment of diseases, such as cancers and infectious diseases.

For example, HSP-complexes may be produced by separating and purifying them from diseased or cancerous tissue. HSP-complexes may also be produced de novo by taling a particular heat shock protein and associating it with a synthetic or naturally occurring peptide or antigen. The de novo production of HSP-complexes would allow the use of HSP-complex vaccines without the need for removing tissue from the patient. U.S. Pat. No. 5,981,706.

HSP-complexes may be derived directly from the diseased tissue or they may be produced de novo by associating known HSPs with naturally occurring or synthetic peptides or other antigens. A method for the de novo production of HSP-complex would allow the large scale production of vaccines without the need for removing tissue from the patient. Also, it permits the large scale production of prophylactic vaccines against a wide variety of diseases by using peptides that represent known oncogenic mutations or peptides representing portions of known viral proteins. Roman et al., “Synthetic Peptides Non-Covalently Bound to Bacterial HSP70 Elicit Peptide Specific T-Cell Responses in vivo.” Immunology. 1996; 88:487-492; “Delayed-type Hypersensitivity Elicited by Synthetic Peptides Complexed with Mycobacterial Tuberculosis Elicited by Synthetic Peptides Complexed with Mycobacterial Tuberculosis HSP70.” Immunology. 1997; 90:52-56.

For example, heat shock proteins produced, isolated and purified in accordance with this invention may be used to produce a HSP-complex de novo. This may be accomplished by binding a particular heat shock protein to a denatured protein matrix and adding a complexing solution comprising a peptide to elute the HSP-complex.

In one embodiment, an ADP-HSP-complex may be produced for use in developing vaccines or immunotherpeutic tools for tumors and infectious diseases. In a preferred embodiment, such complexes may be formed by complexing ADP to the heat shock protein before it is added to the denatured protein matrix. The purified heat shock protein is mixed with ADP and a solution containing the ADP-heat shock protein is added to an agarose-gelatin matrix to bind the heat shock protein to the denatured protein or gelatin. Then a peptide complexing solution containing a peptide complexing agent is added to the column to relase the ADP-HSP-complex as an ADP-HSP-peptide by binding to the peptide binding site on the HSP. U.S. Pat. No. 5,981,706.

Peptides suitable for use as peptide complexing agents include those described in U.S. Pat. No. 5,348,864 (vav mouse oncogene); U.S. Pat. No. 5,320,941 (protein sequence of the mas oncogene and polypeptides derived therefrom); U.S. Pat. No. 5,614,192 (peptides capable of binding to T-cell receptors); and U.S. Pat. No. 5,550,214 (peptides capable of binding HLA-A2 binding cleft and capable of stimulating proliferation of cytotoxic T-lymphocytes), among many others. Typically, the peptide complexing agents may be obtained from purified cell lysates.

Antigens of cancers or infectious diseases can also be obtained by purification from natural sources, by chemical synthesis, or recombinantly, and through in vitro procedures.

### EXAMPLE 4

### Purification of HSP-Complexes

HSP-complexes may be purified as described in Udono et al., “Heat Shock Protein 70-Associated Peptides Elicit Specific Cancer Immunity.” J. Exper. Med. 1993; 178:1391-1396. The purification methods described herein are specifically adapted to purify the HSP-complexes without disrupting the association between the HSP and peptide in the HSP-complex.

Accordingly, in one embodiment, an HSP-complex may be purified by ADP-affinity chromatography. A solution containing the HSP-complexes may be added to a conventional column, such as an agarose gel column, to which ADP has been added to form an ADP matrix. Suitable ADP-agarose columns include those described in U.S. Pat. Nos. 5,114,852; 5,268,465; 5,132,047; and 5,541,095, herein incorporated by reference as if fully set forth herein. Optionally, the HSP-complex solution may be incubated at a temperature of 37 to 50° C. A purifying buffer solution, such as one containing GTP or another non-adenosine containing molecule, may be added to the column to elute other proteins that are loosely associated with the ADP matrix. The HSP-complexes may then be eluted with a buffer solution containing ADP and NaCl. This method has been used with success to purify HSP-complexes, wherein the HSP is HSP70 and the complex is immunologically active. Peng, P. et al. “Purification of immunogenic heat shock protein 70-peptide complexes by ADP-affinity chromatography.” J. Immunol. Methods. 1997; 204:13-21.

In another embodiment, HSP-complexes may be purified in one step using heparin-agarose chromatography. Menoret, A. et al., “Purification of Multiple Heat Shock Proteins from a Single Tumor Sample.” J. Immunol. Methods. 2000; 237:119-130. First, a cell pellet may be homogenized in hypotonic buffer (10 mM NaHCO3, 0.5 mM PMSF, pH 7.0). Second, a 100,000×g supernatant may be obtained. The sample buffer may be changed to the heparin buffer, comprising 20 mM sodium phosphate, 2 mM MgCl2, 0.5 mM DTI, pH 7.2) with PD-10 column (Sephadex G-25, Pharmacia Biotech., Piscataway, N.J.). The sample may be applied at room temperate on a 20-mL Heparin Sepharose (Pharmacia Biotech., Piscataway, N.J.) equilibrated with the heparin buffer and washed. The proteins may be eluted by a linear gradient from 0 to 1 M NaCl in heparin buffer.

In yet another embodiment, a cell pellet obtained by centrifugation may be suspended in 3 volumes of 30 mM sodium bicarbonate, pH 7, 1 mM PMSF. After the cells are allowed to swell for 20 minutes on ice, the cell suspension may be homogenized until most of the cells are lysed. The cell lysate may then be centrifuged to remove cellular debris. Ammonium sulfate may be added to an amount to bring the supernatant to 50% ammonium sulfate saturation.

The solution is may then be centrifuged at 6,000 rpm and the supernatant removed and brought to 70% ammonium saturation and again centrifuged. The pellet from this step may then be saved and suspended in solution of PBS brought to 70% ammonium sulfate saturation. The mixture is centrifuged and the pellet is dissolved in PBS containing 2 mM each of Ca²⁺ and Mg²⁺. The solution may then be mixed with concanavalin A-Sepharose beads, which have previously been equilibrated with 2 mM each Ca²⁺ and Mg²⁺ until the absorbance at 280 nm drops to near zero and is stable. Srivastava, P K. “Purification of Heat Shock Protein-Peptide Complexes for Use in Vaccination against Cancers and Intracellular Pathogens.” Methods: A Companion to Methods in Enzymology. 1997; 12:165-171.

In one embodiment, HSP90-complexes may be purified from the concanavalin-A-unbound fraction. The unbound fraction is first dialyzed against a solution of 20 mM sodium phosphate, pH 7.4, 1 mM EDTA, 250 mM NaCl, 1 mM PMSF. The dialyzed sample may then be centrifuged and the supernatant applied to a Mono Q (Pharmacia) column equilibrated with 20 mM sodium phosphate, pH 7.4, 1 mM EDTA, containing 200 mM NaCl. The bound proteins are eluted with the same buffer by a linear salt gradient up to 600 mM NaCl. Id.

In another embodiment, HSP-70-complexes may be purified by homogenizing a cell pellet in its volume of hypotonic buffer and obtaining the supernatant after centrifugation. The sample buffer may then be changed to Buffer D (comprising 20 mM Tris-acetate, 20 mM NaCl, 15 mM β-mercaptoethanol, 3 mM MgCl₂, 0.5 mM PMSF, pH 7.4) with a PD10 column and applied to an ADP-agaorse column equilibrated with Buffer D. The column may then be washed with Buffer D containing 0.5 M NaCl and then Buffer D alone until no further proteins may be detected by the Bradford protein assay. The column may then be incubated with Buffer D containing 3 mM ADP at room temperature for 30 minutes and then eluted with the same buffer. Id.

### EXAMPLE 5

### Assays to Determine Heat Shock Protein Antibody Levels in Patients as a Diagnostic Marker for Disease

Significantly higher levels of heat shock protein antibodies have been detected in patients with autoimmune and inflammatory diseases. For instance, studies have shown that patients with arthritis manifest T-cell responses to HSP60, suggesting that these patients have antibodies to HSP60. Patients with atherosclerotic lesions were also found to have significantly higher levels of antibodies to HSP60 as compared with healthy patients without atherosclerotic lesions. Xu Q, et al. “Association of Serum Antibodies to Heat Shock Protein 65 with Carotid Atherosclerosis.” Lancet. 1993; 5(6):803-814. Immunoreactivity to HSP60 was also reported to play an important role in the development of insulin-dependent diabetes mellitus. Jones D B, et al. “Heat shock protein 65 as a β cell antigen of insulin-dependent diabetes.” Lancet. 1990; 336:583-585. Patients with insulin-dependent diabetes mellitus were found to have significantly higher frequency and levels of antibodies to HSP60 as compared to healthy patients. Ozawa, Y., et al. “Detection of Autoantibodies to the Pancreatic Islet Heat Shock Protein 60 in Insulin-dependent Diabetes Mellitus.” J. Autoimmun. 1996; 9:517-524.

Antibodies to heat shock proteins have also been detected in patients with diseases, including but not limited to insulin-dependent diabetes mellitus, rheumatoid arthritis, systemic lupus erthematous, temporo-mandibular joint disease, ankylosing spondylitis, and inflammatory bowel diseases. Tishler M et al. “Anti-Heat-Shock Protein Antibodies in Rheumatic and Autoimmune Diseases.” Semin. Arthritis Rheum. 1996; 26(2):558-63. Accordingly, an assay that can determine the level of heat shock protein antibodies in the patient would be an important diagnostic tool for diseases.

Heat shock proteins produced in accordance with the present invention may be used in a diagnostic assay to quantify the level of antibodies present in patient, where a correlation exists between the increased antibody level for the specific heat shock protein and a particular disease. In one general embodiment, a specific heat shock protein is produced and purified in accordance with the present invention. A biological sample, such as patient sera, synovial or other bodily fluid is obtained and exposed to the heat shock protein under conditions that would allow the antibodies to bind to the heat shock protein. The antibodies may then be quantified by enzyme-linked immunosorbant assay (ELISA), radioimmunoassay (RIA), or other immunoas says known to those of ordinary skill in the art.

In one embodiment of the present invention, HSP60 may be used as a diagnostic assay for insulin-dependent diabetes mellitus. Ninety six-well microtitre plates (Nunc, Roskilde, Denmark) may be coated with 50 μl of HSP60 at 4 μg/ml in 0.05 mol/l carbonate buffer (pH 9.6) at 4° C. overnight. The wells may then be washed with a phosphate-buffered saline solution (pH 4.0) containing 0.1% (v/v) Tween-20. A 200 μl solution of phosphate-buffered saline and 1% (w/v) bovine serum albumin is added to each well of the HSP60 coated plates and the non-coated plates and incubated for 1 hour at 37° C. After washing the plates, 50 μl of diluted patient sera (1:20) may be added to each well of the HSP60 coated plates and non-coated plates. The plates may then incubated for 4 hours at 37° C. and again washed. Peroxidase-conjugated goat anti-human IgG Fab₂ at 1:500 dilution may be added to the plates and incubated for 1 hour at 37° C. The plates may then be developed by the addition of orthophenylene diamine (Sigma, St. Louis, Mo.), chromagens, or other chemical detection systems including chemluminescence and chemfluorescence, in the presence of 0.003% (v/v) H₂O₂. The enzyme reaction may be stopped with 3 mmol/l H₂SO₄ or other acid, followed by a measurement of optical density (OD) at 492 nm or other wavelengths appropriate to the wavelength detection chemistry (EIA reader Model 2550 BioRad Laboratories, Richmond, Calif.).

An HSP60 antibody may be derived from the OD₄₉₂ values as a parameter in determining disease state. HSP60 antibody indices may be calculated by (1) subtracting the OD₄₉₂ of the non-coated plates from the OD₄₉₂ of the HSP60 coated plates and (2) dividing this by a value represented by the OD₄₉₂ of HSP60 antibody positive serum from an insulin-dependent diabetic patient minus the OD₄₉₂ of negative serum from a healthy control.

1. A method of manufacturing heat shock proteins comprising a step of initiating a coagulative necrotic process in a tissue.

2. The method of claim 1, wherein the coagulative necrotic process is initiated by subjecting the tissue to energy transfer, incubating the tissue in a bioreactor having a temperature sufficient to induce the production of the heat shock proteins, or exposing the tissue to a condition selected from a group consisting of irradiation, thermal injury, hypoxia and toxin.

3. The method of claim 2, wherein the thermal injury comprises a step of heating the tissue to 60° C. to 80° C.

4. A method of manufacturing heat shock proteins comprising the steps of:
incubating a tissue in a bioreactor having a temperature sufficient to induce the production of the heat shock proteins;
collecting supernatant from the tissue in the bioreactor; and
separating the heat shock proteins contained in the supernatant.

5. A method of manufacturing heat shock proteins comprising the steps of:
heating a tissue sufficient to initiate a coagulative necrotic process in the tissue; cooling the tissue to a room temperature;
incubating the tissue in a growth medium; collecting supernatant from the tissue; and separating the heat shock proteins contained in the supernatant.

6. The method of claim 5, wherein the tissue is heated to a temperature of from 60° to 80° C.

7. The method of claim 5, wherein the tissue is incubated for 48 hours or less.

8. A method of manufacturing heat shock proteins comprising the steps of:
heating the tissue to 60° C. to 80° C. for approximately 10 seconds;
cooling the tissue to a room temperature;
incubating the tissue in a growth media for 48 hours; collecting the supernatant from the tissue; and separating the heat shock proteins contained in the supernatant.

9. The method of any of claims 5 or 8, wherein the tissue is heated for less than 1 minute.

10. The method of any of claims 4, 5 or 8, wherein the tissue is neonatal foreskin.

11. The method of any of claims 4, 5 or 8, wherein the heat shock proteins are separated& by a method selected from the group consisting of centrifugation, electrophoresis and chromatography techniques.

12. The method of claim 11, wherein the chromatographic technique is a method selected from group consisting of gel filtration, ion exchange, chromafocusing, affinity, immunoaffinity, hydrophobic interaction, and reverse phase.

13. The method of claim 11, wherein the heat shock proteins are separated by DE 52 ion-exchange chromatography followed by affinity chromatography on ATP-agarose, a combination of column chromatography and high performance liquid chromatography, DE 52 ion-exchange chromatography followed by a hydroxylapatite column, heparin-agarose chromatography, or gelatin-agarose chromatography.

14. A method of producing an HSP-complex comprising a step of associating the heat shock proteins produced in accordance with the method of claims 1, 4, 5 or 8 with an antigen.

15. The method of claim 14, wherein the antigen is an endogenous antigen, a synthetic antigen, a bacterial antigen, or a viral antigen.

16. The method of claim 14, wherein the antigen is a peptide.

17. The method of claim 14, where the antigen is synthetically produced.

18. The method of claim 14, wherein the antigen is obtained from diseased tissue that is autologous to a patient to whom the HSP-complex is administered.

19. The method of claim 14, wherein the HSP-complex is purified by a chromatographic technique.

20. The method of claim 19, wherein the chromatographic technique is ADP-affinity chromatography or heparin-agarose chromatography.

21. An assay for determining the level of antibodies against heat shock proteins as a diagnostic marker for a disease comprising:
purifying the heat shock proteins produced in accordance with the method of claim 1, 4, 5 or 8;
obtaining a biological sample from an individual;
exposing a biological sample to the heat shock proteins under conditions that would allow antibodies in the biological sample to bind to the heat shock proteins; and quantifying the level of the antibodies in the biological sample.

22. The assay of claim 21, wherein the antibodies are quantified by an immunoassay.

23. The assay of claim 22, wherein the immunoassay is an enzyme-linked immunosorbant assay or a radioimmunoassay

24. The assay of claim 21, wherein the disease is insulin-dependent diabetes mellitus, rheumatoid arthritis, systemic lupus erthematous, temporo-mandibular joint disease, ankylosing spondylitis, or inflammatory bowel disease.

25. A heat shock protein which is manufactured Accordingly to the method of any of claim 1, 4, 5 or 8.

26. A HSP-complex comprising a heat shock protein of claim 25 and an antigen.

27. The HSP-complex of claim 26, wherein the antigen is an endogenous antigen, a synthetic antigen, a bacterial antigen, or a viral antigen.

28. The HSP-complex of claim 26, wherein the antigen is a peptide.

29. The HSP-complex of claim 26, where the antigen is synthetically produced.

30. The HSP-complex of claim 26, wherein the antigen is obtained from diseased tissue that is autologous to a patient to whom the HSP-complex will be administered.

31. (canceled)

32. A vaccine comprising HSP-complex of claim 26.

33. A method for diagnosing a disease in a patient comprising exposing a biological sample from a patient to the heat shock proteins of claim 25 under conditions that would allow antibodies in the biological sample to bind to the heat shock protein and quantifying a level of the antibodies in the biological sample.

34. An assay for diagnosis of disease comprising the heat shock protein of claim 25.
